(19) United States
(12) Patent Application Publication
(10) Pub. No.: US 2020/0247053 A1
(43) Pub. Date: Aug. 6, 2020

(54) A Method, System And Device For Three Dimensional Additive Manufacturing In A Liquid Phase

(71) Applicant: UNIVERSITY COLLEGE DUBLIN, NATIONAL UNIVERSITY OF IRELAND, DUBLIN, DUBLIN (IE)

(72) Inventors: Brian Rodriguez, Dublin, Dublin (IE); Emmanuel Reynaud, Dublin, Blackrock (IE)

(21) Appl. No.: 15/774,376

(22) PCT Filed: Nov. 9, 2016
(86) PCT No.: PCT/EP2016/077055

§ 371 Date: May 8, 2018

(30) Foreign Application Priority Data
Nov. 9, 2015 (GB) 1519730.4

(51) Int. Cl. B29C 64/314; B33Y 10/00; B33Y 30/00; B33Y 70/00; B29C 64/106; B33Y 40/10; C12M 1/12

(52) U.S. Cl. CPC B29C 64/314; B33Y 10/00; B33Y 30/00; C12M 25/14; B29C 64/106; B33Y 40/10; B33Y 70/00

(57) ABSTRACT

A method for fabricating a structure by means of 3D printing, the method comprising the steps of extruding a polymer to form the structure on a platform (8), characterised in that the polymer is extruded, and the structure formed, in a liquid phase (12), and wherein the liquid phase is formulated to modify the structure being fabricated.

### FIELD OF THE INVENTION

The invention relates to a method, system and device for use in material deposition. In particular the invention relates to a method, system and device for fabricating three-dimensional structures.

### BACKGROUND TO THE INVENTION

Three-dimensional (3D) printing is an additive process which involves sequential deposition of material in layers to create a 3D object. This additive process can be done through a wide variety of methods, including granular material deposition (initial depositions of granules are fused into a layer, often via laser sintering. that is then lowered and built further upon), photopolymerization (a vat of UV-reactive liquid is exposed to controlled lighting, causing the liquid to harden and form layers that build into a model) and extrusion deposition (extrusion of material through the extruder opening onto a surface). 3D printing is applicable to many industries on scales ranging from the creation of structures of the order of micrometers to meters.

Fused filament fabrication, also known as extrusion deposition 3D printing, involves the production of the model through the deposition of small particles or beads that immediately fuse into a solid substance. In the majority of cases, the printer contains an X, a Y and a Z stage. The X and Y stages, which are controlled independently by stepper motors, position the extruder head on the platform in X and Y axes. The Z stage controls the position of the extruder in the Z azis or the platform on which the structure is being created. Lowering this platform allows for successive layers to be added to the growing structure. Conversely, the extruder can be raised. The printer heats the extruder, with the temperatures depending on the materials being used.

US2015057786 describes a 3D printer device and methods of use thereof, for printing 3D constructs for use in fabricating tissues and organs. The printer device comprises a means for applying a wetting agent to one or more of: the printer stage; the receiving surface, the deposition orifice, bio-ink, support material, or the printed construct. The wetting agent can be water, tissue culture media, buffered salt solutions, serum, or a combination thereof. The wetting agent is applied simultaneously or substantially simultaneously with or prior to the bio-ink or supporting material being dispensed by the bioprinter.

WO 2014/194180 describes a printing method also, as well as an apparatus for placing cells on a surface comprising: a cell monolayer or biomaterial surface; one or more printing tips; a cartridge for holding said one or more printing tips; and a three-axis motion control system configured to move said cartridge in three dimensions with respect to said cell monolayer or biomaterial surface. A printing platform upon which this is carried out is also described. WO 2014/194180 also appears to describe building a construct using the printer when the printer tips are placed under an aqueous film.

WO 2015/017421 appears to disclose a method for fabricating a structure such as a biological tissue or a tissue engineering scaffold using 3D printing, where the printing method comprises a support bath within which the tissue scaffold is fabricated and which provides divalent cations for crosslinking the printed material. Further, use of a cross-linker concentration in a method for producing rapid prototyping is discussed in EP1517778B; while DE102012100859A discloses a method for producing and printing a 3D structure containing living cells, which may comprise of printing in a high density liquid.

WO 216/019435 appears to disclose an additive manufacturing apparatus comprising a deposition head to extrude a first material into a reservoir containing a second material, wherein a least a portion of the object being manufactured is submerged in the second material. Further, the second material (a fluid) may be recirculated from the reservoir and back again. The document also appears to disclose that the reservoir is temperature controlled and different fluids may be mixed within the extruder.

The problem(s) associated with the 3D printers and methods described in (i) US2015057786, (ii) WO 2014/194180, (iii) WO 2015/017421, (iv) EP 1517778 and (v) DE 102012100859 is that it is not possible to influence or control the structure being the fabricated by manipulating the fabrication environment freely. In (i), the use of a wetting agent is to reduce evaporation during the printing process; in (ii) the method is to provide suitable conditions for cell deposition on a surface; for (iii) the support bath is generally removed by chemical treatment and results in printer clogging; (iv) the method discusses creating a polymer and adding a dye to change colour; and (v) the method relates to extrusion into a dense liquid that provides support to the structure, rather than changing the properties of the structure. The problem with the apparatus of WO 2016/019435 is that the method relates to extrusion into a liquid with a density that provides support to the structure being manufactured, rather than changing the properties of the structure itself. The method only uses one solution which is recirculated for reuse and to keep the level of the solution so that the last layer printed is submerged.

It is an object of the present invention to overcome at least one of the above mentioned problems.

### SUMMARY OF THE INVENTION

To address the issues of the current 3D printers, Applicant has developed a 3D printer which enables a scalable technology platform to fabricate reproducible organ-specific biocompatible 3D biopolymer hydrogels (for example, alginate, collagen, chitosan, fibrin, etc.). The 3D printer described herein will enable the user to create, for example, stem cell niches for stem cell biology and has broad implications for designing better drug screening models, using fewer animals and developing approaches for personal medicines beyond genomic. There is further potential for expansion into flexible electronics, bio-sensing, and the like.

The approach is based on traditional nozzle-injection with multiple syringe capability in order to print different materials, including cells and tubes for, for example, vascularization, in air or liquid. The primary advantage of the approach described herein is that printing in a liquid environment gives a user the possibility to tune the properties of the material—to tailor the physical, chemical and biofunctional properties of the print in real time with micrometre resolution—in order to print reproducible microtissues into a variety of containers, including petri dishes or cell culture dishes, and multiple well-plates (12, 24, 36, 48, 72, 96, etc.) already compatible with many biomedical characterisation tools. Furthermore, the printer of the invention is a temperature-controlled, fluid-exchange system which provides an unlimited range of printing possibilities as the liquid can be modified at will in real time (e.g., temperature, pH, ions, dyes, cross-linkers, drugs, growth factors, enzymes, extracellular matrix components). Moreover, it allows the environment for optimal cell recovery and growth to be tuned during the printing process. 3D printing can be further combined with microcontact lithography to improve resolution and add chemical functionality, using moulds, stamps, etc. The printer footprint has been engineered to be able to place it inside a lamellar flow hood and thus can easily be integrated into cell-culture labs, or it can be made and sold with a bespoke enclosure. The 3D printer described herein also has the potential to create precise, multimaterial scaffolds for complex, hierarchical organotypic tissues.

According to the present invention there is provided, as set out in the appended claims, a fluid exchange system (1) for use in a 3D printer, the fluid exchange system (1) comprising: a platform (8) adapted for supporting a liquid phase (12), an extruder (2) for printing at least one polymer, at least one inflow port (7) for delivering a fluid to the platform (8); at least one outflow port (9) for removing a fluid from the platform (8); and at least one reservoir (40) to supply a fluid to the platform (8) to create the liquid phase (12).

According to the present invention there is provided, as set out in the appended claims, a fluid exchange system (1) for use in a method for fabricating a structure by means of 3D printing according to the method described below, the fluid exchange system (1) comprising: a platform (8) adapted for supporting a liquid phase (12), an extruder (2) for printing at least one polymer, at least one inflow port (7) for delivering a fluid to the platform (8); at least one outflow port (9) for removing a fluid from the platform (8); and at least one reservoir (40) to supply a fluid to the platform (8) to create the liquid phase (12).

Preferably, the platform is temperature regulated.

Preferably, the platform can be heated to 10° C., 15° C., 20° C., 25° C., 30° C., 35° C., 40° C., 45° C., 50° C., 55° C., 60° C., 65° C., 70° C., 75° C., 80° C., 85° C., 90° C., 95° C., 100° C., 105° C., 110° C., 115, 120° C., 125° C., 130° C., 135° C., 140° C., 145° C., 150° C., 155° C., and 160° C. inclusive.

Preferably, the platform can be cooled from room temperature to 10° C.

Preferably, the at least one polymer and the fluid can be delivered to the liquid phase simultaneously.

Preferably, the extruder further comprises an additional inflow pipe for delivery of a second polymer or an additional fluid to mix with the at least one polymer prior to extrusion in the liquid phase.

Preferably, at least one of the parameters of the liquid phase can be further adjusted to control a physical, a biofunctional, chemical and/or a mechanical property of the polymer being printed. More preferably, the at least one parameter of the liquid phase are selected from temperature, pH, ion concentration, dye, cross-linking agent, drug, growth factor, enzyme, extracellular matrix components, or cells.

Preferably, the physical property of the polymer being controlled is selected from viscosity, stiffness, modulus, mechanical properties, elasticity, viscoelasticity, hardness, lubricity, swelling, size, homogeneity, composition, porosity, dimensions, tuneable hydrophilicity, tuneable swellability, resistance to dissolution, tuneable degradability, drug elution, electrical charge of polymer chains (neutral, ionic, ampholytic, zwitterionic), number average molecular weight between cross-links, network mesh size.

Preferably, the chemical property of the polymer being controlled is selected from cross-linking state, synthesis, dissociation, isomerization, oxidation, reduction, decomposition, replacement complexation, polymerisation, catalytic state, photochemical, substitution, elimination, addition.

Preferably, the biofunctional property of the polymer being controlled is selected from inert, antifungal, antibacterial, anti-inflammatory, anti-infective, growth factors, metabolic agents, energy releasing agents (e.g. glucose), hormones, steroids, analgesics, analgesics, anaesthetic, antidepressants, convulsants and anticonvulsants.

Preferably, the mechanical property of the polymer being controlled is selected from elasticity, viscoelasticity, hardness, lubricity, and swelling.

Preferably, the platform is a container, a petri-dish, a cell culture dish, a multi-well plate, a glass slide or any vessel capable being adapted for use in a fluid exchange system with inlets and outlets.

Preferably, the fluid is selected from a buffer, cell culture media, a cross-linking solution, aqueous solutions containing ions, proteins, drugs, oil-based fluids, lipids, glycerol.

According to the present invention there is provided, as set out in the appended claims, a 3D printer comprising the fluid exchange system as described above.

According to the present invention there is provided, as set out in the appended claims, a method for fabricating a structure by means of 3D printing, the method comprising the steps of extruding at least one polymer to form the structure on a platform, characterised in that the at least one polymer is extruded, and the structure formed, in a liquid phase, and wherein the liquid phase is configured to modify the physical, chemical, mechanical and biofunctional properties of the structure being fabricated by controlling a fluid exchange in the liquid phase in real time during fabrication.

Preferably, the liquid phase is contained within the platform area upon which the structure is fabricated.

Preferably, the liquid phase is formulated to comprise at least one component selected from a buffer, a cell culture media, a cross-linking solution, aqueous solutions containing ions, proteins, drugs, and the like.

Preferably, at least one parameter of the liquid phase can be modified in real-time by actively replacing or adding a component to the liquid phase to modify the structure being fabricated. More preferably, the parameters are selected from temperature, pH, ion concentration, dye, cross-linking agent, drug, growth factor, enzyme, extracellular matrix components, or cells.

Preferably, the liquid phase can be further modified by the addition of prokaryotic cells and/or eukaryotic cells.

Preferably, the fabricated structure is selected from a hydrogel, a biological tissue, a microtissue, a hierarchical organotypic tissue, a scaffold, a biomaterial, an organic material, a composite material, a nanomaterial, an encapsulated material, a drug delivery particle, a drug eluting material, a dye, a fluorescent label, a quantum dot, a cell, a diatom.

Preferably, the platform is a container, a petri-dish, a cell culture dish, a multi-well plate, a glass slide or any vessel capable being adapted for use in a fluid exchange system with inlets and outlets.

Preferably, the base of the platform is pre-conditioned with a sacrificial priming skirt. Preferably, the platform further comprises a layer or base upon which the sacrificial priming skirt is printed, the layer or base is comprised of a glass slide or plate, a plastic sheet, sandpaper, filter paper, polylactic acid (PLA), a further petri dish or cell culture dish, or the like.

Preferably, the polymer is one or more selected from a monomer, copolymer, homopolymer, multipolymer, natural or synthetic, such as a hydrogel, alginate, collagen, chitosan, fibrin, poly(ethylene glycol), synthetic hydrogel, hyaluronic acid, block copolymers.

Preferably, the extruder further comprises a solution selected from a buffer, cell culture media, a cross-linking solution, aqueous solutions containing ions, proteins, drugs, etc..

Preferably, at least two polymers are combined in the extruder.

Preferably, the extruder is a syringe, a syringe with a plunger, a syringe pump or other suitable pump device, a cartridge, a tube.

Preferably, the extruder is a syringe having a needle gauge selected from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30.

Preferably, the shape of the opening of the extruder can control the shape of the print from the extruder.

Preferably, the shape of the opening of the extruder is configured to print a structure selected from a solid tube, a hollow tube, a star-shaped extrusion, a square a circle, a polygon.

Preferably, the method further comprises providing a means for controlling delivery of a crosslinking agent to the liquid phase or extruder.

Preferably, the method further comprises a means for controlling fluid exchange in the liquid phase.

Preferably, the method further comprises a means for controlling an extrusion rate of the polymer.

Preferably, the method is further combined with microcontact lithography and/or photopolymerisation.

The fluid exchange system is compatible with a 3D printer and allows the cross-linking solution to be changed while a structure is being printed, thus enabling, for example, the user to tailor the physical, chemical and biofunctional properties of the structure, and introduce physical, chemical and biofunctional property gradients. Tailoring the chemical properties can be achieved by influencing the structure being fabricated as it responds to changes in pH, the ionic strength of the buffer or media, the solvent composition of the same and the molecular species being used. The method and fluid exchange system described herein allows a user to print stable structures into a container when the pumps for the liquid phase and extruder are running.

The advantages of the method and fluid exchange system described herein are that:
1. Printing in liquid is better for any hydrogel as it maintains their water content and properties.
2. Liquid phase can be tailored for any hydrogel but also other polymers (specific chemistry).
3. Liquid phase can be used to polymerise or improve polymerisation.
4. Liquid phase can contain cross-linkers.
5. Liquid phase can contains proteins, dyes or any element needed to modify, improve chemically or physically the polymer being printed.
6. Physical parameters of the liquid phase can be modified (pH, temperature, etc.) to improve, chemically or physically, the polymer being printed.
7. The liquid phase properties can be tailored during the printing process per layer but also within the same layer (gradient).
8. The polymer can be modified within the extruder (for example, a syringe) to alter the physical, chemical and/or mechanical properties of the polymer.
9. The polymer can be combined with other polymers within the syringe/extruder.
10. The end of the extruder can be modified to print many different forms of structure, for example, a solid tube, a hollow tube, star-shaped etc.

It is possible to print on typical substrates used in cell culture such as glass, plastic, metals, and also on other hydrogels, biopolymers, biomaterials and mono- or multi-cell layers.

Both ion concentration and temperature mediated cross-linking, can be implemented through software or manual control during the extrusion via control of extrusion rate and printing speed. The shape of the extruded material further depends on the physical parameters of the syringe tip (gauge, flat or bevelled end, opening shape, etc.). All parameters of printing and fluid exchange are controllable by software, script or manually during the printing process. The invention relates to an integrated fluid exchange 3D printer.

The present invention has a heating stage and fluid-exchange system, and has been used to print multilayer 3D structures of, for example, alginate-gelatin in liquid. The composition of the liquid is important for ensuring that the 3D structure forms; printing in air or water does not result in a stable structure, however, printing in a precisely mixed crosslinking solution does. The Applicants have further demonstrated that the method and fluid exchange system described herein permits printing in multiple types of containers, including multiple-well plates, large baths, integrated macro-to-micro fluidics, petri dishes, etc.

The system described herein may be retrofitted to commercially available printers.

One advantage of the fluid exchange system described herein is that optimization of alginate-gelatin and oxidized alginate-gelatin scaffolds, for example, can be achieved by controlling crosslinking, ratios, print speed, extrusion speed, nozzle size, viscosity, etc. during the printing process.

The advantage of pre-conditioning the platform by printing a sacrificial priming skirt thereon is that it improves adhesion and stability of the structure being printed.

In the specification, the term “priming skirt” should be understood to mean a layer of polymer or hydrogel deposited or printed on the platform surface and which is an outline of the structure being fabricated. The skirt also helps to ensure that the printed polymer or hydrogel is securely attached to the surface of the platform or layer or base and also stabilises the resulting structure. The skirt primes the extruder by beginning the flow of polymer or gel through the extruder. It also allows time for the polymer or hydrogel to adhere to the platform, which can often take several seconds, before the printing of the structure, construct or scaffold begins. The priming skirt can be printed in air or solution and the structure being fabricated is then printed in solution. The priming skirt facilitates the attachment and stabilisation of the fabricated structures, which allows the structures themselves to be moved or removed from the platform (or from within the printing apparatus, or a layer or base or petri dish, or whatever it is printed on) or from one location to another on the printing apparatus. For example, one can fabricate individual structures on a support platform primed with a sacrificial skirt and move the structures to a 96-well plate, and test molecules in each of the wells on the separate structures. This allows one to move the structures to a plate rather than moving the platform in the printing apparatus to test the molecules. Alternatively, one could print directly into a multi-well plate with (or without, depending on the polymer and conditions) a priming skirt, and then move the multi-well plate from the platform and place it, for example, in an incubator. It allows the user to pick up the soft object (the printed structure) and move it directly from the platform or layer or base without damaging the printed structure.

In the specification, the term “liquid phase” should be understood to mean extrusion of the polymer into solution.

In the specification, the term “modified in real-time” should be understood to mean that parameters can be modified during the extrusion process, such as the feedback loop between the fluid exchange and the extrusion step. The modifications can be controlled by a computer system either via a pre-set program or manually by the user during the printing process. For example, the flow rate of the extruder or the rate of buffer flow into the platform, the temperature of the extruder or the liquid within the platform, the speed and direction of the extruder in X, Y and Z planes, the addition of agents (physical, chemical, biofunctional) to the extruder or platform (e.g. liquid phase).

In the specification, the term “formulated to modify the structure” should be understood to mean modification of the physical, chemical, and biofunctional properties of the structure through fluid exchange. For example, any solution aqueous or otherwise which can modify the physical chemical or biofunctional properties or the extruded material or composite material including but not limited to buffer, cell culture media, a cross-linking solution, serum, aqueous solutions containing ions, proteins, drugs, etc. or a combination thereof or serve as a support structure. The addition of a liquid phase with high steroid concentration will lead to a print layer loaded with steroid that can be later used by cells to grow or migrate faster at this given position, e.g. the increase in the crosslinking concentration in the fluid phase will increase the cross-linked state of the extruded polymer, leading to a higher density print at the following layers and can give migration cues to cells or nerve endings. The increase in the crosslinking concentration in the fluid phase will increase the cross-linked state of the extruded polymer leading to a higher density print at the following layers and can give migration cues to cells or nerve endings or create a local density that can improve the print (stiffness).

In the specification, the term “modified structure” should be understood to mean and include modification of the composition of the structure, modification of the, or a, physical parameter of the structure (such as, for example, by controlling the rate of crosslinking, the mechanical properties, topography, roughness of the structure), chemical modification (such as, for example, adding functional groups to the polymers making up the structure; controlling the cross-linking and surface chemistry of the structure), and biofunctional modifications (such as, for example, the additional of proteins, drugs, growth factors etc. to the structure).

In the specification, the term “microcontact lithography” should be understood to mean a form of soft lithography that uses the relief patterns on a master polydimethylsiloxane (PDMS) stamp to form patterns of self-assembled monolayers (SAMs) of ink or proteins on the surface of a substrate through conformal contact, as in the case of micro contact or nanotransfer printing. Its applications are wide-ranging, including microelectronics, surface chemistry and cell biology.

In the specification, the term “polymer” should be understood to mean any natural or synthetic polymer commonly used in any combination and also as composite materials incorporating particles, nanomaterials, etc. polyethylene glycol; synthetic hydrogel, hyaluronic acid or any material and scaffolds that are extrudable, biocompatible, with limited by-products and stable.

In the specification, the term “hydrogel” or “hydrogels” can be interchangeable with “polymer” and should be understood to mean a network of natural, synthetic or hybrid polymer chains that are hydrophilic and/or hydrophobic. A hydrogel can be a homopolymer (a single polymer chain), a copolymer (two polymer chains), or a multipolymer (a plurality of different polymer chains). The polymers may be selected from alginate, collagen, fibrin, silk, lysozyme, synthetic hydrogel, poly(ethylene glycol), Matrigel® (a gelatinous protein mixture secreted by Engelbreth-Holm-Swarm (EHS) mouse sarcoma cells produced and marketed by Corning Life Sciences), calmodulin, elastin-like polypeptides; polysaccharides such as hyaluronic acid (HA), agarose, dextran, chitosan; protein/polysaccharide hybrids such as collagen/HA, laminin/cellulose, gelatin/chitosan and fibrin/alginate; deoxyribonucleic acid (DNA); degradable and non-degradable synthetic polymers such as the block copolymers polylactide-block-poly(ethylene glycol)-block-polylactide (PLA-PEG-PLA) and poly(ethylene glycol)-block-polylactide-block-poly(ethylene glycol) (PEG-PLA-PEG) diacrylates, disulfide-containing polyethylene glycol diacrylates (PEG(SS)DA), (hydroxyethyl)methacrylate (HEMA), acrylamide (AAm), acrylic acid (AAc), (N-isopropylacrylamide) (NIPAm), Poly(N-isopropylacrylamide) (PNIPAm) and poly(ethylene glycol) methacrylate (mPEGMA); natural/synthetic hybrids such as PEG-modified heparin, dextran, HA, fibrinogen, albumin; PNIPAm-modified collagen, chitosan and alginate; other synthetic peptide-modified proteins or polysaccharides; poly(vinyl alcohol) (PVA) modified natural polymers.

In the specification, the term “flow barrier” should be understood to mean a physical barrier composed of grids, meshes or a pegboard having different mesh, pore or hole sizes. The number and size of the pores used would depend on the speed (pump pressure) of the flow or density of the fluid. For example, if a fluid is delivered from the inlet port at high pressure, it would be necessary to use a flow barrier to break the flow of fluid, release the pressure and ensure an even distribution of the fluid on the platform. This “flow barrier” is similar to a baffle system which is used to reduce turbulent flow in a fluid. For example, to achieve a higher pressure the flow barrier may have a reduced pore size so as to reduce maximum velocity. To achieve a low pressure flow, the flow barrier may have larger pores. Alternatively, the barriers may be removed altogether. In baffle systems, the height of the barrier, the thickness of barrier and the porosity can be changed. The flow barrier may be made from any material that is suitable to safely support (e.g. inert, stable) the fluid being used in the liquid phase of the printing process. For example, the flow barrier may be composed of poly(methyl methacrylate) (PMMA), or polymers having similar physical properties when set. In the system described herein, the function of the flow barrier is to control the inflow and the outflow of the fluid from the inlet port to the liquid phase and from the liquid phase to the outflow port, while avoiding any fluid-based disturbance of the printing process (e.g. drag, drift, lateral displacement). The advantage of the flow barrier is that it allows for a smooth exchange of fluid into and out of the liquid phase while printing, in between layer prints, or any other related printing steps.

### BRIEF DESCRIPTION OF THE DRAWINGS

The invention will be more clearly understood from the following description of an embodiment thereof, given by way of example only, with reference to the accompanying drawings, in which:

FIG. 1 illustrates a fluid exchange system of the claimed invention.

FIG. 2 illustrates a plan view of the fluid exchange system of FIG. 1, without the extruder head visible.

FIG. 3 illustrates one example of a single layer print using the fluid exchange system described herein wherein the single layer print was performed in a crosslinking solution.

FIG. 4 illustrates one example of a multilayer print using the fluid exchange system described herein. In this case, 8 layers of alginate-gelatin have been printed in a crosslinking solution to create an intact three dimensional object.

FIG. 5A and 5B illustrate that the biochemical properties of the print using the fluid exchange system described herein can be altered from a normal polymer print (5A) and incorporation of magenta dye (5B).

FIG. 6A and 6B illustrate one example of altering the physical properties of the printed material, in this case, by changing the syringe gauge (6A: 14 gauge tip; 6B: 27 gauge tip).

### DETAILED DESCRIPTION OF THE DRAWINGS

### Materials and Methods

### Printer Equipment

Any ‘do it yourself’ kit for assembling a bioprinter is readily available, such as the Ultimaker Original™. 3D printers generally consist of a platform with an adjustable bed, X, Y and Z axes run by stepper motors, an extruder head and an extruder which pushes the filament through a heated nozzle. The extruder head sits on two metal bars attached to the X and Y axes which control the movement around the bed. The bed platform sits on a threaded bar (the Z axis) which controls the Z positioning during printing. One embodiment of a printer used herein was constructed using these parts and the extruder modified for use with a syringe. It should be understood that the system of the invention can be used in other printers, such as RepRap (replicating rapid prototype) printers and 2D printers. The RepRap printers are 3D printers that are an open design, released under a free software license (the GNU General Public License), and use an additive manufacturing technique called fused filament fabrication (FFF) to lay down material in layers. It will also be possible to inject a polymer with a syringe in a photopolymerisation-based 3D printer and exchange the resin bath with different polymers (e.g. to change colours) to affect the printed structure.

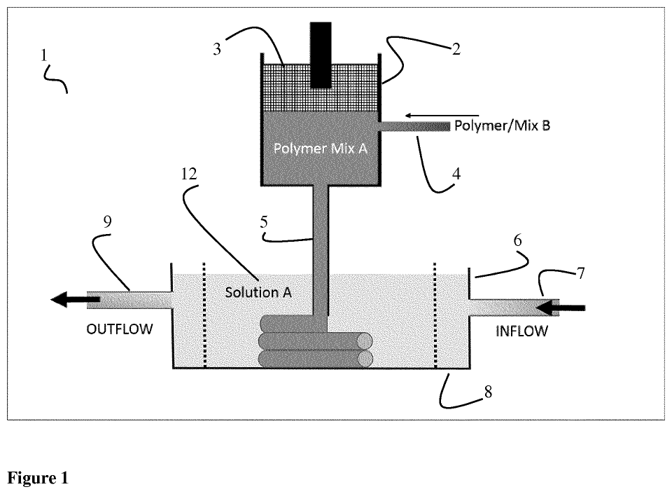

Turning now to FIG. 1, there is illustrated a fluid exchange system of the present invention. Specifically, FIG. 1 illustrates a plan view of a fluid exchange system of the present invention and is generally referred to by reference numeral 1. The fluid exchange system comprises an extruder head 2 and a platform 6. The extruder head 2 generally having an inlet port (compartment) 3, an outlet port (compartment) 5 and an optional inlet port (compartment) 4. The platform 6 generally comprises an inflow port 7, a container 8 and an outflow port 9. The extruder head 2 stores a polymer A/polymer mix B prior to extruding the polymer A/polymer mix B through the outlet port 5 and into a liquid phase (printing area) 12 held in the container 6. The optional inlet port 4 supplies a polymer B/polymer mix B (or an additional fluid) to the extruder head 2. The polymer stored in the extruder head 2 is generally a hydrogel-forming polymer as described herein.

The position of the extruder head 2 is controlled using stepper motors commonly found in all 3D printers. In the majority of cases, the 3D printer contains an X, Y and Z stage. The X and Y stages, which are controlled independently by stepper motors, position the extruder head 2 over the platform 6. The z stage controls the position of the extruder head 2 or the platform 6 on which the structure is being created. Lowering the platform 6 allows for successive layers to be added to the growing structure. The 3D printer heats the extruder head 2, with the temperatures being deployed depending on the materials being used. The extrusion can be controlled by, but is not limited to, pushing on a syringe using a stepper motor or syringe pump, etc., and other methods commonly used in the art. Alternatively, the flow system described herein can be used in a 3D printer where the extruder head is fixed and the X, Y, Z stages control the movement of the platform.

Turning now to FIG. 2, there is illustrated a plan view of the fluid exchange system 1 of FIG. 1. The inflow port 7, controlled by pump A, enters a buffer zone 16 and the outflow port 9, controlled by pump B, exits a buffer zone 16′. Separating the liquid phase (printing area) 12 from the buffer zones 16, 16′ is a flow barrier 18, 18′. The flow barrier 18, 18′ defines a buffer zone 20, 20′, respectively, which controls the flow of fluid through the system 1 and optimises fluid exchange without affecting the printing procedure. The flow barrier 18, 18′ comprise apertures 22 of defined size or meshes to limit current and flow disturbances when the fluid flows through from the inflow port 7 to the liquid phase 12, thus preventing deleterious effects on the quality of the object being printed.

The pumps A, B are software and feedback controlled by a computer 30, which drives the printing process. This permits for a tighter control of the fluid exchange and, for example, the hydrogel printing process. The two pump system associated with the fluid exchange system 1 can push the fluid through the inflow port 7 or pull the fluid through the outflow pump 9, depending of the properties of the fluid and the rate of fluid exchange and flow required for the object being printed. For example, simultaneous actions of pumps A, B can permit a fast fluid exchange or a more controlled and constant flow across the liquid phase 12 for a regular replenishment of cross linkers during an entire printing process. This two tier pump system is also very efficient for establishing gradients.

The buffer zones 16 and flow barrier 18 can be referred to as Filter A and Filter B, respectively, while buffer zone 16′ and flow barrier 18′ can be referred to as Filter C and Filter D, respectively. These four filter layers establish the four buffer zones 16, 16′, 20, 20′. The Filters A to D can be removed or their dimensions and properties (for example, the size of the apertures 22) tailored at will to ensure an efficient exchange of fluid across the printing area. The Filters A to D can be made of materials that are inert and stable in the fluids being exchanged, including cellulose, ceramic, plastic, nylon, polycarbonate, polytetrafluorethylene (PTFE), polyamide or any other filtering-type material known in the art, but also can be in the form of grids made from materials such as, for example, metals (stainless steel, titanium, aluminium, etc.), polyvinyl chloride (PVC), polylactic acid (PLA, polylactide), Poly(methyl methacrylate) (PMMA), or other materials known in the art.

The inflow and outflow of fluid from the inlet port 7 to the outflow port 9 is controlled by using any type of pump or gravity-based liquid exchange devices known in the art. The inflow and outflow of fluid can be simultaneously or sequentially activated by use of either both or only one single flow line (e.g. inflow port 7 only). One of the aims of the process of the exchange of fluid in the fluid exchange system 1 described herein is to provide a smooth transition of the liquid phase during the printing steps. In addition the fluid exchange system 1 can be used to wash off any remnants, debris or excess of unpolymerised polymers and fluids from the platform 6 following completion or otherwise of the printing process. In the case of an active 3D printing process, i.e., when material is being extruded or modified in real time, the inflow port 7 and outflow port 9, as well as the buffer zones 16, 16′, are optimized for the type of liquid phase for every parameter (e.g. temperature, density, volume of fluid) to allow an optimal exchange of fluid in the liquid phase without disturbing the ongoing 3D printing in progress. For example, for active printing, low flow and low turbulence conditions would be required, depending on the speed of the print. The speed of the print is optimized by adjusting the flow rate and the flow barrier characteristics which controls the flow rate. The flow barrier would likely have small pores in this case to provide a low flow environment. Depending on the type of fluid being used in the liquid phase, natural diffusion of a highly concentrated solution (for example, a crosslinking agent) across the fluid of the liquid phase present on the platform can be preferred, while an inflow at higher pressure could be used to wash off any excess of the precedent active fluid in the liquid phase prior to inflow of a new liquid phase for a subsequent step.

### Printing

### Material Extrusion

The extrusion of the polymer/hydrogel from the extruder head 2 per se may be driven by two separate motors. One motor capable of pushing a plunger within one inlet port (compartment) of the extruder head 2 to extrude a polymer/hydrogel; each motor capable of pushing a plunger within a respective inlet port (compartment) of the extruder head 2 where there are two inlet ports, each inlet port storing a polymer/hydrogel solution; and/or one or both motors adapted to exert a force to mix or extrude one or both of the polymer/hydrogel solutions through the extruder head 2 in a movement as described above.

The extrusion of the polymer/hydrogel from the extruder head 2 may be driven by a motor capable of pushing a plunger of a syringe. The polymer/hydrogel will be extruded from the end of the syringe tip and laterally constrained by the gauge of the opening. The influence of gauge opening on the dimensions of the extruded material is illustrated in FIG. 6A and 6B. The object in FIG. 6A was extruded from a 14 gauge tip whereas the object in FIG. 6B was extruded from a 27 gauge tip.

### Heating

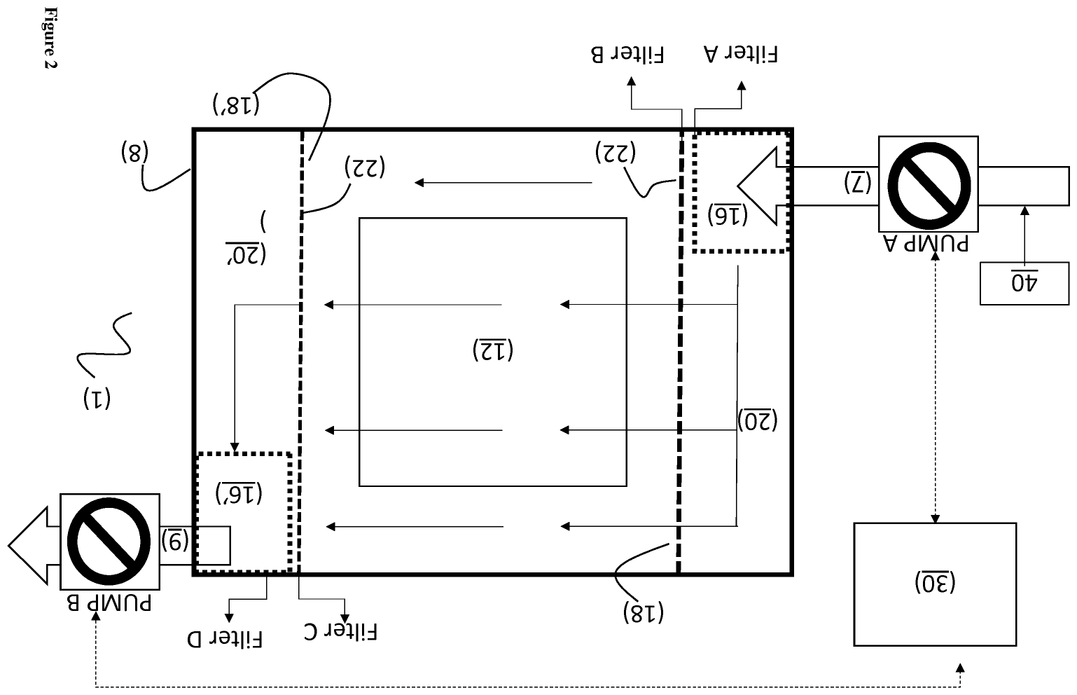

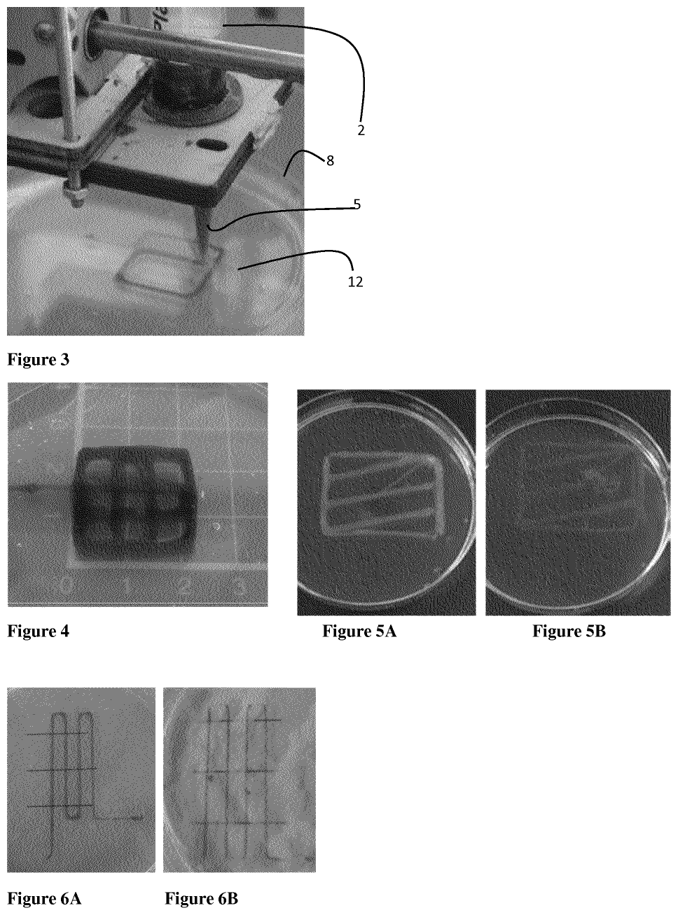

The extruder head 2 of the fluid exchange system 1 can be either a syringe extrusion system (as depicted in FIG. 1) or a pump, as well as a traditional fusion deposition system, once the fluid has been chosen and tested accordingly. Fusion deposition systems generally direct the successive layering of hot plastic polymer that are fused to each other using a hot end head. The deposition of layer after layer of hot polymer ensures the fusion of each layer to one another, allowing for fusion/deposition and ensuring the integrity of the final product. However, in the case of cross linking and polymerisation of polymer (hydrogel), this approach requires the presence of an optimal polymerisation environment to allow the crosslinking of previous layer with the newly deposited layer. If not, there will not be enough inter layer bonds to ensure the final integrity of the 3D printed structure. An example of successful single and multilayer prints are shown in FIG. 3 and FIG. 4, respectively. An example of the use of a priming skirt is shown in FIG. 3. FIG. 5A and FIG. 5B illustrate that the biochemical properties of the print can be altered through, in this example, the incorporation of magenta dye (FIG. 5B). As an alternative to the extruder head 2 as depicted in FIG. 1, the extruder head 2 can optionally comprise two separate inlet ports having a common outlet port, which would allow mixing of components such as polymer mix A and/or polymer/polymer mix B with cross-linkers and other components. Both the inlet port 3 (which can also be referred to as “compartment(s)”) and the extruder head 2 (including any needle) can be heated. A voltage controlled heater element can be used to heat the extruder 2 or inlet port 3 (compartment(s)).

The heater element can be flat and rigid or flexible and conformal, e.g., it can be a ‘Kapton insulated flexible heater’ or a ‘Flexible silicone heater’ or other heater known in the art.

The heater element can be wrapped around the extruder head 2 (for example, the body of a syringe/tube/compartment) and the thermal conductivity can be improved by using encasing the extruder head 2 in a thermally conductive holder in contact with the heater.

The voltage can be supplied using the voltage outputs of a 3D printer, or using a Raspberry pi/Arduino/external voltage source. A thermocouple can be used to monitor the temperature. A proportional-integral-differential (PID) controller can be used as a feedback loop to maintain temperature independently for each of the two separate inlet ports (compartments) or the extruder head 2.

Heating of the extruder head 2 or the two separate inlet ports (compartments) allows the viscosity of the polymer/hydrogel to be reduced, ensuring the polymer/hydrogel does not get stuck, and can be extruded uniformly. It allows the mechanical properties of the polymer/hydrogel to be tailored. Application of heat allows the use of polymers with higher rigidity to be extruded than would normally be possible at room temperature. These polymers can also aid in providing mechanical stability to the print, even before interaction with the cross-linking solution in the bath.

### Movement

The extruder head 2 is generally controlled to move in X, Y and Z stages, the same as any other extruder head in a 3D printer and the movement mechanism can be taken from those printers known in the art. In general, the mechanism consists of a rail system, belts, stepper motors, and is generally referred to by those skilled in the art as a drive train system. This is described here: http://reprap.org/wiki/Category:DriveTrains. Basically, voltage is applied to a stepper motor that causes rotation, which is translated into independent linear motion along the X, Y and Z stages. The mechanism of movement is the same as that of a Computer Numerical Control (CNC) router, which is a computer controlled cutting machine related to the hand held router used for cutting various hard materials, such as wood, composites, aluminium, steel, plastics, and foams, and is familiar to those skilled in the art of 3D printing.

### FLUID BATH

### Heating

Heating of the platform 6 provides a route for tailoring the mechanical, biofunctional, and chemical properties of the print, including stabilization of the structure being fabricated. A heating element can be embedded in the support structure of the platform 6 or integrated in the container 8 itself to heat the liquid phase 12.

### Cooling

Cooling the platform 6 to a temperature that is below room temperature can be achieved by placing the system 1 in a cold room (a refrigerated room), in a refrigerator, using a heat exchange system, ice bath, or cooling by air or water flow, Peltier element or other method known in the art.

### Exchange Means for Controlling Fluid Exchange in the Liquid Phase

The liquid phase 12 of the platform 6 can be changed before/during or in between extrusion steps using, for example, the pumps A, B which move fluid from a reservoir 40 via the inflow port 7 to the container 8 and from the container 8 thought the outflow port 9 via pump B to a waste container. The pump A, B is voltage controlled and software controlled via the computer 30. The liquid phase 12 is equipped with buffer zones 16, 16′, 20, 20′ using dividers 18, 18′ with a plurality of apertures 22 of defined size or meshes as well as specific nozzles to limit vortex formation and avoid effects on the quality of the extrusion.

The fluid exchange process is a software controlled and feedback circuit controlled by the computer 30 during the extrusion process. The computer 30 is able to adapt the fluid exchange and the extrusion steps as needed. The fluid exchange platform 8 can be linked to more than one reservoir to allow for mixing of components prior to injection in the printing area 12, allowing for multiple combinations and changes during one single print.

In addition, the liquid phase 12 can be used as a pH neutralization step at the end of a print. The liquid phase 12 can also be used as a washing system to clean the platform 6 prior to a new print, or to remove or polish the final print (e.g. via surface modification chemistry).

In the specification the terms “comprise, comprises, comprised and comprising” or any variation thereof and the terms include, includes, included and including” or any variation thereof are considered to be totally interchangeable and they should all be afforded the widest possible interpretation and vice versa.

The invention is not limited to the embodiments hereinbefore described but may be varied in both construction and detail.

1. A method for fabricating a structure by means of 3D printing, the method comprising the steps of extruding at least one polymer to form the structure on a platform, characterised in that the at least one polymer is extruded, and the structure formed, in a liquid phase, and wherein the liquid phase is configured to modify the physical, chemical, mechanical and biofunctional properties of the structure being fabricated by controlling a fluid exchange in the liquid phase in real time during fabrication.

2. A method according to claim 1, wherein the liquid phase is contained within the platform area upon which the structure is fabricated.

3. A method according to claim 1, wherein the liquid phase is formulated to comprise at least one component selected from a buffer, a cell culture media, a cross-linking solution, aqueous solutions containing ions, proteins, drugs, and the like.

4. A method according to claim 1, wherein at least one parameter of the liquid phase can be modified in real-time by actively replacing or adding a component to the liquid phase to modify the structure being fabricated.

5. A method according to claim 1, wherein at least one parameter of the liquid phase can be modified in real-time by actively replacing or adding a component to the liquid phase to modify the structure being fabricated and wherein the parameters are selected from temperature, pH, ion concentration, dye, cross-linking agent, drug, growth factor, enzyme, extracellular matrix components, or cells.

6. A method according to claim 1, wherein the liquid phase can be further modified by the addition of prokaryotic cells and/or eukaryotic cells.

7-8. (canceled)

9. A method according to claim 1, wherein the base of the platform is pre-conditioned with a sacrificial priming skirt.

10. A method according to claim 1, wherein the base of the platform is pre-conditioned with a sacrificial priming skirt and wherein the platform further comprises a layer or base upon which the sacrificial priming skirt is printed, the layer or base being comprised of a glass slide or plate, a plastic sheet, sandpaper, filter paper, polylactic acid (PLA), a further petri dish or cell culture dish, or the like.

11. (canceled)

12. A method according to claim 1, wherein the extruder further comprises a solution selected from a buffer, cell culture media, a cross-linking solution, aqueous solutions containing ions, proteins, drugs, etc..

13. A method according to claim 1, wherein at least two polymers are combined in the extruder.

14-21. (canceled)

22. A fluid exchange system (1) for use in a method for fabricating a structure by means of 3D printing according to claim 1, the fluid exchange system (1) comprising: a platform (8) adapted for supporting a liquid phase (12), an extruder (2) for printing at least one polymer, at least one inflow port (7) for delivering a fluid to the platform (8); at least one outflow port (9) for removing a fluid from the platform (8); and at least one reservoir (40) to supply a fluid to the platform (8) to create the liquid phase (12).

23. A fluid exchange system according to claim 22, wherein the platform is temperature regulated.

24-26. (canceled)

27. A fluid exchange system according to claim 22, wherein the extruder further comprises an additional inflow pipe for delivery of a second polymer or an additional fluid to mix with the at least one polymer prior to extrusion in the liquid phase.

28. A fluid exchange system according to claim 22, wherein at least one of the parameters of the liquid phase can be further adjusted to control a physical, a biofunctional, chemical and/or a mechanical property of the polymer being printed.

29. (canceled)

30. A fluid exchange system according to claim 22, wherein at least one of the parameters of the liquid phase can be further adjusted to control a physical, a biofunctional, chemical and/or a mechanical property of the polymer being printed; and wherein the physical property of the polymer being controlled is selected from viscosity, stiffness, modulus, mechanical properties, elasticity, viscoelasticity, hardness, lubricity, swelling, size, homogeneity, composition, porosity, dimensions, tuneable hydrophilicity, tuneable swellability, resistance to dissolution, tuneable degradability, drug elution, electrical charge of polymer chains (neutral, ionic, ampholytic, zwitterionic), number average molecular weight between cross-links, network mesh size.

31. A fluid exchange system according to claim 22, wherein at least one of the parameters of the liquid phase can be further adjusted to control a physical, a biofunctional, chemical and/or a mechanical property of the polymer being printed; and wherein the chemical property of the polymer being controlled is selected from cross-linking state, synthesis, dissociation, isomerization, oxidation, reduction, decomposition, replacement complexation, polymerisation, catalytic state, photochemical, substitution, elimination, addition.

32. A fluid exchange system according to claim 22, wherein at least one of the parameters of the liquid phase can be further adjusted to control a physical, a biofunctional, chemical and/or a mechanical property of the polymer being printed; and wherein the biofunctional property of the polymer being controlled is selected from inert, antifungal, antibacterial, anti-inflammatory, anti-infective, growth factors, metabolic agents, energy releasing agents (e.g. glucose), hormones, steroids, analgesics, analgesics, anaesthetic, antidepressants, convulsants and anticonvulsants.

33. A fluid exchange system according to claim 22, wherein at least one of the parameters of the liquid phase can be further adjusted to control a physical, a biofunctional, chemical and/or a mechanical property of the polymer being printed; and wherein the mechanical property of the polymer being controlled is selected from elasticity, viscoelasticity, hardness, lubricity, and swelling.

34. (canceled)

35. A fluid exchange system according to claim 22, wherein the fluid is selected from a buffer, cell culture media, a cross-linking solution, aqueous solutions containing ions, proteins, drugs, oil-based fluids, lipids, glycerol.

36. A 3D printer comprising the fluid exchange system of claim 22.
